(19) United States
(12) Plant Patent Application Publication
(10) Pub. No.: US 2017/0094855 P1
(43) Pub. Date: Mar. 30, 2017

(54) Mandarin tree named `N40W-6-3'

(50) ;

(71) Applicant: Florida Foundation Seed Producers, Inc., Marianna, FL (US)

(72) Inventor: Jude W. Grosser, Winter Haven, FL (US)

(21) Appl. No.: 14/756,644

(22) Filed: Sep. 28, 2015

(51) Int. Cl. A01H 5/00

(52) U.S. Cl. PLT/202

(57) ABSTRACT

A new hybrid mandarin tree particularly distinguished by producing delicious and seedless tangerine fruit from October to December in Central Florida is disclosed.

Latin name of the genus and species of the plant claimed: Citrus reticulata Blanco hybrid

Variety denomination: ‘N40W-6-3’

### BACKGROUND OF THE INVENTION

The present invention relates to a new and distinct, nearly seedless variety of hybrid mandarin (tangerine) for the fresh citrus market named ‘N40W-6-3’. ‘N40W-6-3’ was selected from a population of 50 plants grown from irradiated budwood (cobalt 60, Vindicator Co., Mulberry, Fla.) from ‘Snack’ (unpatented), a seedy mandarin hybrid of unknown parentage. Irradiation treatment of budwood is known to induce mutations that, in rare instances, cause a permanent genetic change in a variety to greatly reduce or eliminate seed in fruit, without otherwise altering the integrity of the variety. ‘N40W-6-3’ was first asexually propagated by grafting to experimental rootstock Orange #14 (unpatented) and planted in Lake Alfred, Fla., in 2011. Asexually propagated trees of ‘N40W-6-3’ have remained true-to-type to the original selection through successive generations.

### SUMMARY OF THE INVENTION

‘N40W-6-3’ produces small to medium seedless (or nearly seedless) fruit with a unique and robust sweet flavor, and should compete favorably with the ‘Fall-Glo’ mandarin hybrid in the fall fresh mandarin market. Harvest can begin in October, but the fruit reaches maximum flavor and quality around the last two weeks of November. The fruit is generally seedless, although an occasional seed can be found, unlike the original variety ‘Snack’, which averages more than 16 seeds per fruit. Fruit of ‘N40W-6-3’ can be peeled, however it should not be considered a zipper-skinned tangerine selection. Fresh juice obtained from fruit of ‘N40W-6-3’ also has outstanding flavor, but fruit quality data is only available from trees grown on ‘Swingle’ (unpatented) citrumelo rootstock. The exceptional juice flavor did not hold up during processing. A portion of the first-year crop can exhibit a rougher rind and lower juice content, although this diminishes with subsequent crops. ‘N40W-6-3’ makes a small compact tree when grown on ‘Swingle’ citrumelo rootstock. Trees appear to yield adequately, although no formal yield data is available at this time. Fruit size is generally smaller with heavy crops.

### BRIEF DESCRIPTION OF THE DRAWINGS

‘N40W-6-3’ is illustrated by the accompanying photographs which show the plant's form, foliage, and fruit. The colors shown are as true as can be reasonably obtained by conventional photographic procedures. Tree and leaf photographs were taken in November of 2010, and fruit photographs were taken in January of 2011.

FIG. 1—Shows whole and cut fruit of ‘N40W-6-3’.

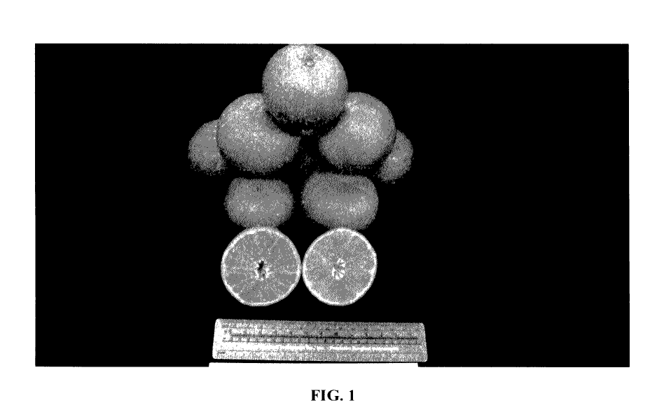

FIG. 2—Shows a tree of ‘N40W-6-3’, showing plant habit, including foliage and fall fruit.

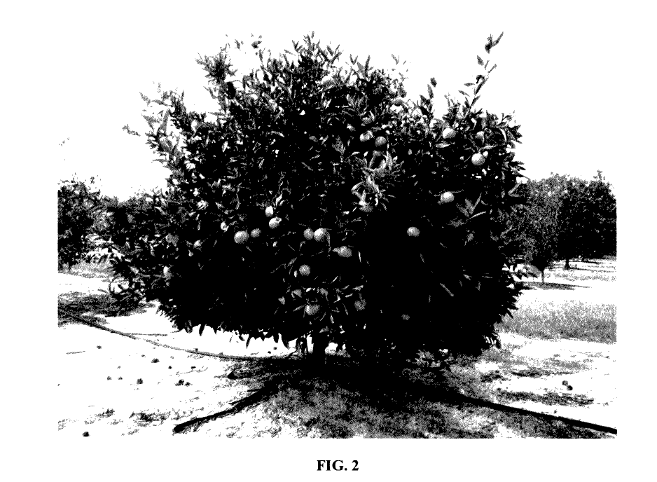

FIG. 3—Shows a close-up of ‘N40W-6-3’ twigs and mature leaves.

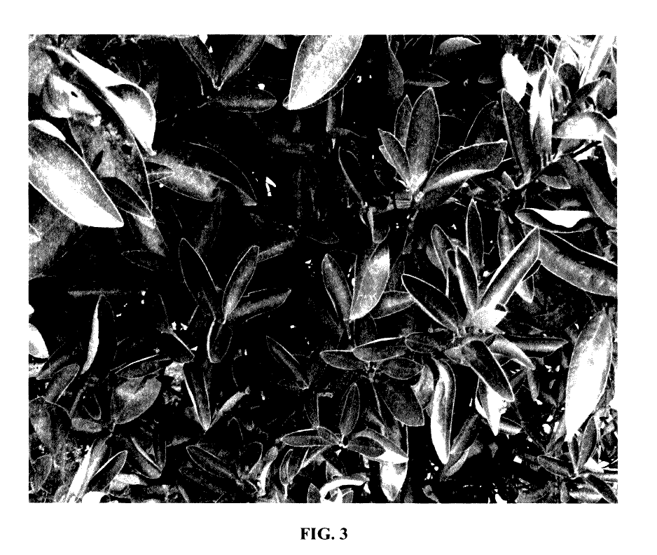

FIG. 4—Shows mature fruit of ‘N40W-6-3’ demonstrating typical rind color and shape.

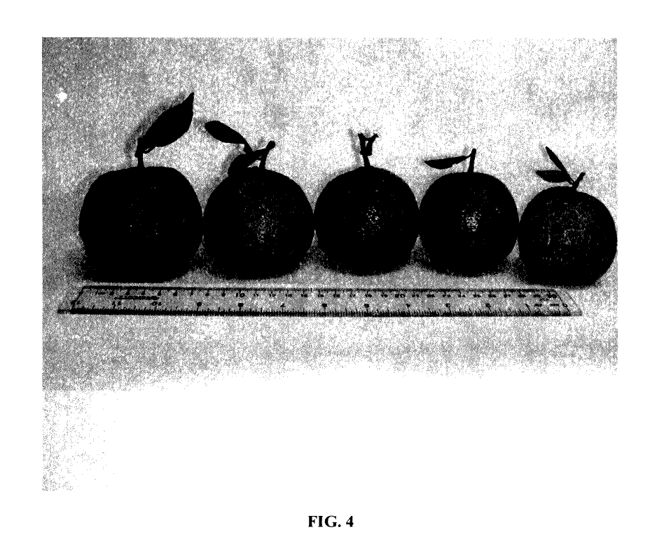

### DETAILED BOTANICAL DESCRIPTION

The following detailed description sets forth the distinctive characteristics of ‘N40W-6-3’. The present botanical description is that of the variety grown on 7-year-old trees growing on ‘Swingle’ rootstock in Lake Alfred, Fla. The colors (except those in common terms) are described from the R.H.S. Colour Chart published by The Royal Horticultural Society in London (second edition), in association with the Flower Council of Holland.
Phenotypic Description of Citrus reticulata Blanco ‘N40W-6-3’
Classification:
   Botanical.—Citrus reticulata Blanco.
   Common name.—Mandarin hybrid or Tangerine.
Parentage:
   Female parent.—Unknown.
   Male parent.—Unknown.
Tree:
   Ploidy.—Diploid.
   Size.—Compacted, medium-small size with heights of 1.8 to 1.9 m, currently unpruned.
   Height.—1.8 to 1.9 m unpruned.
   Tree spread.—1.8 to 2.0 m.
   Vigor.—Moderately vigorous growth; normally growing shoots of 0.2 to 0.3 m, typically from new flush on current tree.
   Density.—Canopy is quite dense. Branches are quite compact on the tree canopy, resulting in a small tree.
   Form.—The shape of the tree is obloid with upright branches. Branches exhibit very little drooping with fruit.
   Growth habit.—Compact and upright.
Trunk:
   Trunk diameter.—14.6 cm at 30 cm above the ground.
   Trunk texture.—Smooth.
   Trunk bark color.—RHS 197A (greyed-green), irregularly striated with RHS N189A (greyed-green) and RHS 189A (greyed-green).
Branches:
   Crotch angle.—Major branches form a 40- to 45-degree crotch angle.
   Branch length.—1.2 to 1.6 m.
   Branch texture.—Relatively smooth and without thorns or spines.
   Branch color (shoots from previous flush, hardened, and 4 to 5 mm in diameter).—RHS 138A (green).
Leaves:
   Size (lamina average).—Length: 67 mm Width: 30 mm L/W ratio: 2.2.
   Thickness.—Regular and average for commercial mandarin hybrids.
   Type.—Simple.
   Shape.—Elliptical.
   Apex.—Slightly retuse.
   Base.—Acute to sub-obtuse.
   Margin.—Entire; smooth and very slightly undulate.
   Surface.—Upper surface: Glabrous Lower surface: Medium veins that are pinnately netted.
   Color.—Upper surface (adaxial): RHS 138A (green) Lower surface (abaxial): RHS 137D (green).
   Petiole.—Shape: Brevipetiolate (shorter than leaf lamina); junction between petiole and lamina is articulate Width (petiole wing): Very narrow Shape (petiole wing): Obovate Length: 6 to 8mm Width: 1 to 1.5 mm Color: RHS 137A (green).
Flowers and flower buds:
   Type.—Hermaphroditic.
   Flower blooming period.—First bloom observed: Late March, in Central Florida Full bloom observed: Early April, in Central Florida.
   Flower petals.—Number: 5 Color: RHS 155B (white) with RHS 150A (yellow-green) spots distributed at the back side of the petal Length: 13 mm Width: 4 mm Calyx: Small to medium sized.
   Fragrance.—Fragrant compared to other citrus blossoms.
   Reproductive organs.—Fertility: Self-fertile Stamen number: 3 per petal Anther color: RHS 12B (yellow) Anther filament length: Shorter relative to stigma Pollen amount: Moderately abundant Pollen color (general): RHS 12C (yellow).
Fruit:
   Size.—Uniform.
   Height.—60 to 65 mm.
   Width.—65 to 75 mm; some fruits reach 85 mm.
   Average weight (per individual fruit).—136.6 g.
   Shape.—Round.
   Shape (cross-section).—Round.
   Apex.—Truncated and slightly depressed.
   Apex cavity diameter.—8-10 mm, usually filled with pithy material.
   Base.—Flat with wrinkles, occasionally some fruit have a very short neck.
   Harvesting period.—October to December.
Skin:
   Adherence.—Albedo (mesocarp) to flesh (endocarp) is medium-low, compared to other commercial hybrid varieties; not a zipper-skin, but can be peeled cleanly.
   Thickness.—2.0 to 3.0 mm.
   Texture.—Pitted.
   Color.—Flavedo (epicarp): Ranges from RHS 25A (orange) to RHS 28A (orange-red) Albedo (mesocarp): RHS 24C (orange).
   Stylar end.—Closed.
   Rind oil cell density.—68 to 74 oil cells per square centimeter.
Flesh:
   Number of segments.—10 to 12 segments per fruit on average.
   Segment walls.—Thin with sufficient strength to maintain integrity as separated.
   Color.—RHS 25B (orange).
   Juice.—Abundant.
   Juice color.—41.5.
   Texture.—Firm.
   Vesicles.—Length: 6 to 15 mm Diameter (thickness): 3 to 4 mm.
   Eating quality (varies from season to season).13 Excellent unique, sweet, but robust flavor (data from fruit harvested Nov. 22, 2010). Fruit generally reaches legal maturity in October, but reaches peak flavor around the end of November. Juice has excellent flavor when fresh squeezed, although flavor does not hold up during processing.
   Soluble solids (average).—12.03.
   Acidity (average).—0.59.
   Ratio.—20.39.
Seeds:
   Type.—Polyembryonic.
   Number.—Ranges from 0 to 1. Most of the fruit contain 0 seeds or undeveloped seed. Occasionally some fruit contain 1 seed (compared to the original ‘Snack’ selection, which contains approximately 16 seeds per fruit).
   Shape.—Normal seed are mostly obclavate and club shaped.
   Size.—Length: 13.0 mm Width: 6.0 mm.
   Seed coat color.—Outer Surface: RHS 159C (orange-white) and smooth Inner surface: RHS 165C (greyed-orange) Cotyledon color: RHS 138C (green).
Resistance to disease: Resistant to citrus canker. Trees are susceptible to HLB (Huanglongbing or citrus greening disease), but show reasonable tolerance and sustained production if grown under an optimized nutrition program, particularly CRF (controlled-release fertilizer) containing high levels of manganese and boron.

What is claimed is:

1. A new and distinct mandarin tree called ‘N40W-6-3’ as illustrated and described herein.
